(19) United States
(12) Patent Application Publication
(10) Pub. No.: US 2010/0100135 A1
(43) Pub. Date: Apr. 22, 2010

(54) BONE FIXATION DEVICE HAVING AN EXPANDABLE ANCHOR

(76) Inventor: Christopher U. PHAN, San Leandro, CA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/255,064

(22) Filed: Oct. 21, 2008

(51) Int. Cl. A61B 17/04; A61B 17/58

(52) U.S. Cl. 606/301; 606/300; 606/104

(57) ABSTRACT

An apparatus includes a first elongate member and a second elongate member. The first elongate member has a proximal end portion that includes a threaded portion, and a distal end portion that includes a retention portion. The retention portion of the first elongate member is configured to deform when moved from a first configuration to a second configuration. At least a central portion of the second elongate member is disposed within the first elongate member. The second elongate member has a distal end portion that includes an actuation portion configured to deform the retention portion of the first elongate member from the first configuration to the second configuration.

### BACKGROUND

The invention relates generally to medical devices and procedures. More particularly, the invention relates to apparatus and methods for inserting a bone fixation device into bone tissue.

Bone fixation devices, such as, for example, bone screws, staples, pins, and/or clamping mechanisms, can be used in various medical procedures. For example, known bone screws can be used to repair fractured bone tissue by clamping adjacent portions of the bone tissue together. Known bone screws can also be used to stabilize and/or limit the movement of bone tissue. For example, some known bone screws can be used as a part of a spinal fixation procedure.

In some procedures, for example, a facet screw can be inserted across the facet joint of the spinal column to fuse and/or limit the motion of the facet joint. Such known procedures often include inserting the bone screw via a medial-to-lateral approach. Said another way, such known procedures often include inserting the bone screw adjacent a midline axis of the spinal column and moving the bone screw in a lateral direction across the facet joint. Such known procedures also often include anchoring the distal end portion of the bone screw within the facet and/or the pedicle of inferior level via a threaded portion. Such known procedures can include, for example, translaminar facet screw fixation, which includes inserting a facet screw from the base of the spinous process on the contralateral side and through the lamina to traverse the facet joint in a plane perpendicular to the joint surfaces. Facet screws can also be inserted using a transfacet approach, which involves inserting a bone screw via a midline incision or an ipsilateral incision.

During such known procedures, however, the bone screw and/or the tool used to insert the bone screw can often interfere with surrounding anatomy. For example, when inserting a facet screw via a midline incision according to the transfacet approach, the tool used to place the screw may undesirably contact the spinous process, causing a less than optimal trajectory of the bone screw, thereby complicating the procedure. Such interference is more likely to occur with higher lumbar levels and/or in smaller patients. Moreover, some known bone screws that are threadedly anchored within a portion of a bone structure can, at times, fail to provide sufficient pull-out strength when anchored within the bone structure.

Thus, a need exists for improved bone fixation devices and procedures for inserting bone fixation devices into bone tissue.

### SUMMARY

Apparatus and methods for inserting facet anchoring pins are described herein. In some embodiments, an apparatus includes a first elongate member and a second elongate member. The first elongate member has a proximal end portion that includes a threaded portion, and a distal end portion that includes a retention portion. The retention portion of the first elongate member is configured to deform when moved from a first configuration to a second configuration. At least a central portion of the second elongate member is disposed within the first elongate member. The second elongate member has a distal end portion that includes an actuation portion configured to deform the retention portion of the first elongate member from the first configuration to the second configuration.

### BRIEF DESCRIPTION OF THE DRAWINGS

FIGS. 1 and 2 are block diagrams of a medical device according to an embodiment in a first and second configuration, respectively.

FIGS. 3 and 4 are perspective views of a medical device according to an embodiment in a first and second configuration, respectively.

FIGS. 5 and 6 are perspective views of the medical device of FIGS. 3 and 4, respectively, showing a nut coupled to a portion of the medical device.

FIGS. 7 and 8 are cross-sectional views of the medical device of FIGS. 3-6 taken along line X-X in FIGS. 5 and 6, respectively.

FIG. 9 is a flow chart of a method according to an embodiment.

FIGS. 10-13 are perspective views showing the medical device of FIGS. 3 and 4 disposed within a body using a method according to an embodiment.

FIGS. 14 and 15 are cross-sectional views of a medical device according to an embodiment in a first and second configuration, respectively.

FIGS. 16 and 17 are cross-sectional views of a medical device according to an embodiment in a first configuration and a second configuration, respectively.

FIG. 18 is a cross-sectional view of a medical device according to an embodiment in a second configuration.

FIG. 19 is a cross-sectional view of a medical device according to an embodiment in a second configuration.

FIG. 20 is a partial cross-sectional view of a medical device according to an embodiment.

FIGS. 21 and 22 are schematic illustrations of a medical device according to an embodiment in a first configuration and a second configuration, respectively.

FIG. 23 is a perspective view of a medical device according to an embodiment in a first configuration.

FIGS. 24 is a cross-sectional view of the medical device of FIG. 23 in the first configuration.

FIGS. 25 and 26 are perspective views of the medical device of FIG. 23 in a first configuration and a second configuration, respectively.

### DETAILED DESCRIPTION

Apparatus and methods for inserting facet anchoring pins are described herein. In some embodiments, an apparatus includes a first elongate member and a second elongate member. The first elongate member has a proximal end portion that includes a threaded portion, and a distal end portion that includes a retention portion. The retention portion of the first elongate member is configured to deform when moved from a first configuration to a second configuration. At least a central portion of the second elongate member is disposed within the first elongate member. The second elongate member has a distal end portion that includes an actuation portion configured to deform the retention portion of the first elongate member from the first configuration to the second configuration.

In some embodiments, an apparatus includes a first elongate member, a second elongate member, and a nut threadedly coupled to the first elongate member. The first elongate member includes a retention portion having a first size when in a first configuration and a second size when in a second configuration. The second elongate member has at least a portion disposed within the first elongate member. The second elongate member has an actuation portion configured to move the retention portion of the first elongate member from the first configuration to the second configuration when the portion of the second elongate member is moved within the first elongate member.

In some embodiments, a method includes inserting a bone fixation device into a body. The bone fixation device includes an elongate member and a nut threadedly coupled to the elongate member. At least a portion of the elongate member is disposed within a passageway defined by a bone tissue, which can include, for example, one or more bone structures (e.g., vertebra). A retention portion of the elongate member is deformed such that a surface of the retention portion of the elongate member is in contact with a first external surface of the bone tissue. The nut is moved relative to the elongate member in a first direction such that a surface of the nut is in contact with a second external surface of the bone tissue. Optionally, in some embodiments, the nut is moved relative to the elongate member in a second direction opposite the first direction.

As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the words “proximal” and “distal” refer to the direction closer to and away from, respectively, an operator (e.g., surgeon, physician, nurse, technician, etc.) who would insert the medical device into the patient, with the tip-end (i.e., distal end) of the device inserted inside a patient's body first. Thus, for example, the end of a medical device first inserted inside the patient's body would be the distal end, while the opposite end of the medical device (e.g., the end of the medical device being operated by the operator) would be the proximal end of the medical device.

The term “parallel” or is used herein to describe a relationship between two geometric constructions (e.g., two lines, two planes, a line and a plane, two curved surfaces, a line and a curved surface or the like) in which the two geometric constructions are substantially non-intersecting as they extend substantially to infinity. For example, as used herein, a line is said to be parallel to a curved surface when the line and the curved surface do not intersect as they extend to infinity. Similarly, when a planar surface (i.e., a two-dimensional surface) is said to be parallel to a line, every point along the line is spaced apart from the nearest portion of the surface by a substantially equal distance. Two geometric constructions are described herein as being “parallel” or “substantially parallel” to each other when they are nominally parallel to each other, such as for example, when they are parallel to each other within a tolerance. Such tolerances can include, for example, manufacturing tolerances, measurement tolerances or the like.

The terms “perpendicular”, “orthogonal”, and/or “normal” are used herein to describe a relationship between two geometric constructions (e.g., two lines, two planes, a line and a plane, two curved surfaces, a line and a curved surface or the like) in which the two geometric constructions intersect at an angle of approximately 90 degrees within at least one plane. For example, as used herein, a line is said to be normal to a curved surface when the line and the curved surface intersect at an angle of approximately 90 degrees within a plane. Two geometric constructions are described herein as being, for example, “perpendicular” or “substantially perpendicular” to each other when they are nominally perpendicular to each other, such as for example, when they are perpendicular to each other within a tolerance. Such tolerances can include, for example, manufacturing tolerances, measurement tolerances or the like.

It should be understood that the references to geometric constructions are for purposes of discussion and illustration. The actual structures may differ from geometric ideal due to tolerances and/or other minor deviations from the geometric ideal.

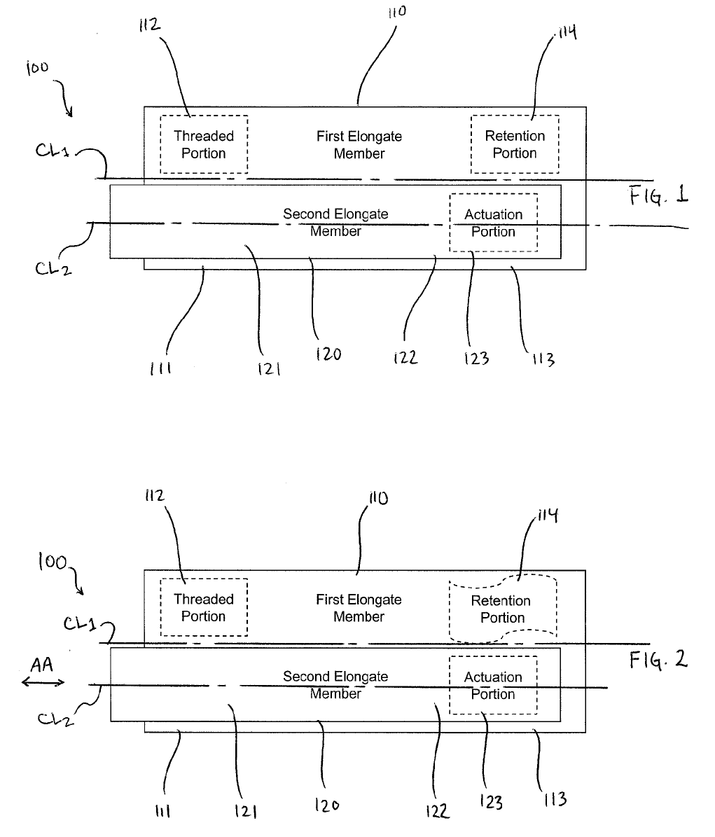

FIGS. 1 and 2 are system block diagrams of a bone fixation device 100 according to an embodiment in a first and a second configuration, respectively. The bone fixation device 100 includes a first elongate member 110 and a second elongate member 120. The first elongate member 110 has a proximal end portion 111 and a distal end portion 113, and defines a longitudinal center line CL₁. The proximal end portion 111 of the first elongate member 110 includes a threaded portion 112. In some embodiments, the threaded portion 112 can be configured to be threadedly coupled to a nut (not shown in FIGS. 1 and 2). In other embodiments, the threaded portion 112 can include self-tapping threads. In such embodiments, the proximal end portion 111 of the first elongate member 110 can be threadedly disposed within bone tissue without requiring a threaded passageway within the bone tissue. The distal end portion 113 of the first elongate member 110 includes a retention portion 114 configured to limit movement of the bone fixation device 100 within a bone tissue (not shown in FIGS. 1 and 2), as described in more detail herein.

The second elongate member 120 has a central portion 121 and a distal end portion 122, and defines a longitudinal center line CL₂. At least the central portion 121 of the second elongate member 120 is disposed within the first elongate member 110. In some embodiments, for example, the central portion 121 of the second elongate member 120 can be disposed within an opening, groove and/or channel (not shown in FIGS. 1 and 2) defined by the first elongate member 110. In some embodiments, the central portion 121 of the second elongate member 120 can be substantially surrounded by the first elongate member 110. In other embodiments, the central portion 121 of the second elongate member 120 can be partially enclosed and/or surrounded by the first elongate member 110. For example, in some embodiments, the central portion 121 of the second elongate member 120 can be disposed within a C-shaped channel (not shown in FIGS. 1 and 2) defined by the first elongate member 110.

Although the central portion 121 of the second elongate member 120 is shown as being disposed within the first elongate member 110 such that the longitudinal center line CL₂ of the second elongate member 120 is offset from and substantially parallel the longitudinal center line CL₁ of the first elongate member 110, in other embodiments, the central portion 121 of the second elongate member 120 can be disposed within the first elongate member 110 such that the longitudinal center line CL₂ of the second elongate member 120 is substantially coaxial with the longitudinal center line CL₁ of the first elongate member 110. In yet other embodiments, the central portion 121 of the second elongate member 120 can be disposed within the first elongate member 110 such that the longitudinal center line CL₂ of the second elongate member 120 is non-parallel to the longitudinal center line CL₁ of the first elongate member 110.

The distal end portion 122 of the second elongate member 120 includes an actuation portion 123 configured to move the retention portion 114 of the first elongate member 110 between a first configuration and a second configuration. More particularly, the actuation portion 123 of the second elongate member 120 is configured to deform the retention portion 114 of the first elongate member 110 to move the bone fixation device 100 from the first configuration to the second configuration, as described in more detail below. The actuation portion 123 can include any suitable mechanism for deforming the retention portion 114 of the first elongate member 110. For example, in some embodiments, the actuation portion 123 can include a mechanical feature (e.g., a shoulder, a protrusion, a detent or the like) configured to contact a portion of the retention portion 114 to deform the retention portion 114. In other embodiments, the actuation portion 123 can include a hydraulic and/or pneumatic mechanism configured to exert a force on a portion of the first elongate member 110 to deform the retention portion 114. In yet other embodiments, the actuation portion 123 can include an electro-mechanical device configured to deform the retention portion 114.

In some embodiments, the second elongate member 120 can be a rigid member configured to transfer a force and/or movement from the proximal end portion 121 of the second elongate member 120 to the actuation portion 123 of the second elongate member 120. For example, in some embodiments, the second elongate member 120 can be a rod configured to transfer rotational motion, longitudinal motion, a torque and/or a force to the actuation portion 123 of the second elongate member 120. In other embodiments, the second elongate member 120 can be a flexible member configured to transfer a force and/or movement from the proximal end portion 121 of the second elongate member 120 to the actuation portion 123 of the second elongate member 120. For example, in some embodiments, the second elongate member 120 can be a flexible cable, tether or the like.

The bone fixation device 100 can be moved between a first configuration (FIG. 1) and a second configuration (FIG. 2). When the bone fixation device 100 is in the first configuration, the retention portion 114 of the first elongate member 110 has a first size, orientation and/or shape. The size, orientation and/or shape of the retention portion 114 when the bone fixation device 100 is in the first configuration is such that movement of the bone fixation device 100 within a bone structure (not shown in FIGS. 1 and 2) is not limited by the retention portion 114.

The bone fixation device 100 can be moved from the first configuration to the second configuration by moving the second elongate member 120 as shown by the arrow AA in FIG. 2. In this manner, the actuation portion 123 of the second elongate member 120 deforms the retention portion 114 to move the bone fixation device 100 from the first configuration to the second configuration. When the bone fixation device 100 is in the second configuration, the retention portion 114 of the first elongate member 110 has a second size, orientation and/or shape different from the first size, orientation and/or shape described above. The size, orientation and/or shape of the retention portion 114 when the bone fixation device 100 is in the second configuration is such that movement of the bone fixation device 100 within a bone structure (not shown in FIGS. 1 and 2) is limited by the retention portion 114.

Although the second elongate member 120 is shown as moving in a direction substantially parallel to the longitudinal center line CL₂ of the second elongate member 120, in other embodiments, the second elongate member 120 can move in any suitable manner to move the bone fixation device 100 between the first configuration and the second configuration. For example, in some embodiments, the second elongate member 120 can move in a direction non-parallel to the longitudinal center line CL₂ of the second elongate member 120. In other embodiments, the second elongate member 120 can rotate about the longitudinal center line CL₂ of the second elongate member 120. In yet other embodiments, the second elongate member 120 can rotate about any axis non-parallel to the longitudinal center line CL₂ of the second elongate member 120. In still other embodiments, the second elongate member 120 need not move to move the bone fixation device 100 between the first configuration and the second configuration. For example, in some such embodiments, the actuation portion 123 can deform the retention portion 114 by supplying an electrical current to the retention portion 114. In other such embodiments, the actuation portion 123 can deform the retention portion 114 by increasing the temperature of the retention portion 114, thereby causing the size, shape and/or configuration of the retention portion 114 to change. For example, in some embodiments, the retention portion 114 can be constructed from a shape-memory alloy, such as, for example Nitinol, having a first shape when at a first temperature and a second shape when at a second temperature.

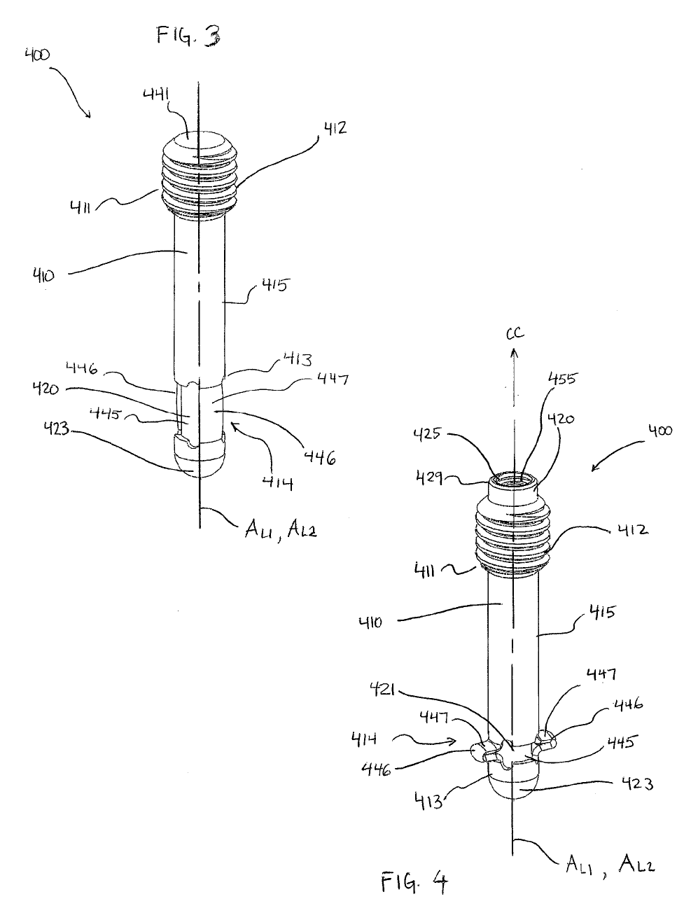

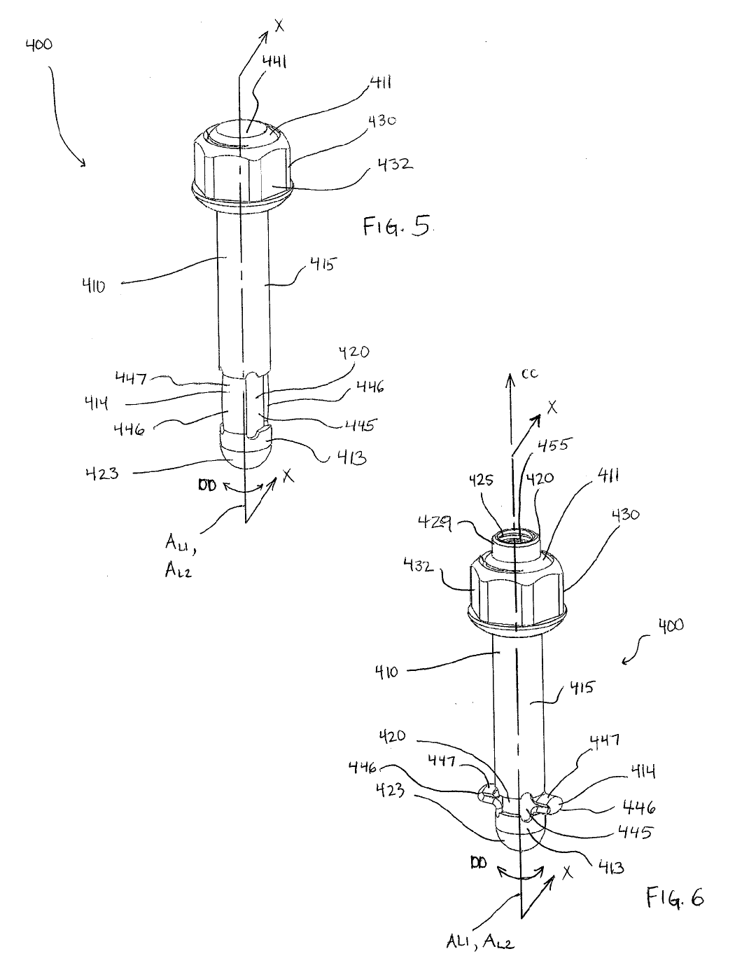

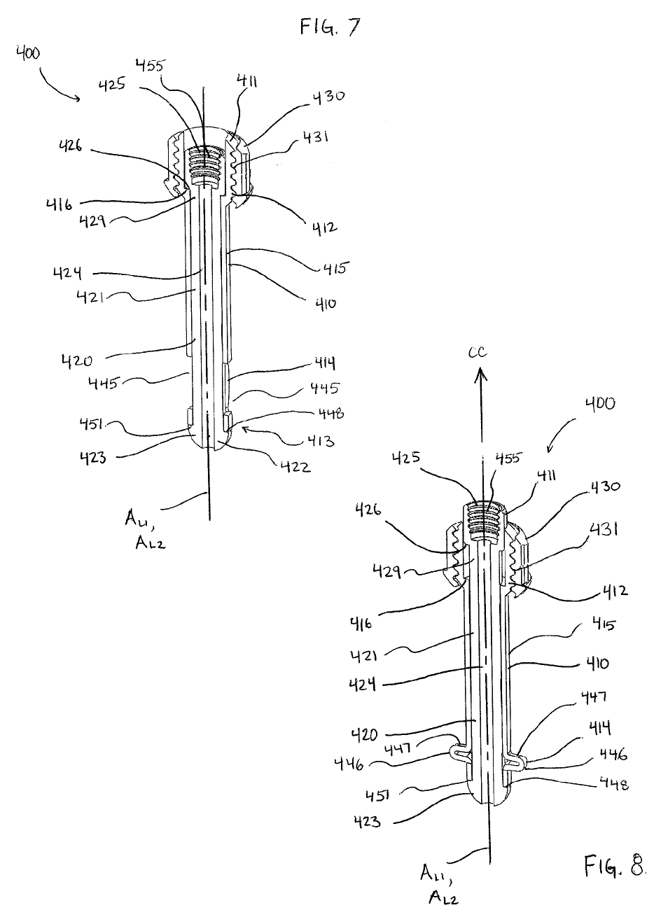

FIGS. 3-8 show a bone fixation device 400 according to an embodiment. More particularly, FIGS. 3 and 5 show perspective views of the bone fixation device 400 in a first configuration, and FIGS. 4 and 6 show perspective views of the bone fixation device 400 in a second configuration. FIGS. 7 and 8 are cross-sectional perspective views of the bone fixation device 400 taken along line X-X in FIGS. 5 and 6, respectively. The bone fixation device 400 includes a first elongate member 410, a second elongate member 420, and a nut 430 (shown in FIGS. 5-8) threadedly coupled to the first elongate member 410.

The first elongate member 410 includes a proximal end portion 411, a distal end portion 413, and a central portion 415 between the proximal end portion 411 and the distal end portion 413. The first elongate member 410 defines a longitudinal axis A_{L1} and a lumen 441 that is substantially centered about the longitudinal axis A_{L1} (see FIGS. 7 and 8). As described in more detail herein, at least a central portion 421 of the second elongate member 420 is disposed within the lumen 441 (best seen, for example, in FIGS. 7 and 8). The lumen 441 has a first portion having a first diameter, and a second portion having a second diameter less than the first diameter, forming a shoulder 416 where the two portions meet.

The proximal end portion 411 of the first elongate member 410 includes a threaded portion 412. More particularly, the threaded portion 412 includes male threads on an external surface of a proximal end portion 411 of the first elongate member 410. The threaded portion 412 of the first elongate member 410 corresponds to (i.e., has the same nominal size and thread pitch) the threaded portion 431 of the nut 430. In this manner, the nut 430 can be threadedly coupled to the first elongate member 410.

The distal end portion 413 of the first elongate member 410 includes a retention portion 414, which defines a pair of collapsible portions 446. Similarly stated, the side wall of the retention portion 414 defines two openings 445 arranged such that the collapsible portions 446 are spaced apart substantially equally about the circumference of the first elongate member 410. Although the retention portion 414 is shown as including two portions 446, in other embodiments, the retention portion can include any number of collapsible members and/or portions disposed in any arrangement (e.g., symmetrical or asymmetrical).

The second elongate member 420 includes a proximal end portion 429, a distal end portion 422, and a central portion 421 between the proximal end portion 429 and the distal end portion 422. The second elongate member 420 defines a longitudinal axis A_{L2} and a lumen 424, each of which is substantially centered about the longitudinal axis A_{L1}. At least the central portion 421 of the second elongate member 420 is disposed within the lumen 441 of the first elongate member 410.

The proximal end portion 429 of the second elongate member 420 includes an engagement portion 425 (best shown in FIGS. 7 and 8) configured to engage an insertion tool (not shown in FIGS. 3-8). The engagement portion 425 defines female threads disposed within the lumen 424 such that an insertion tool can be threadedly coupled to the second elongate member 420. In other embodiments, the engagement portion 425 can include any suitable feature for engaging an insertion tool. For example, in some embodiments, the engagement portion 425 can include male threaded portion to be disposed within a corresponding female threaded portion from an insertion tool. In other embodiments, the engagement portion 425 can include a hexagonal shaped recess configured to receive a corresponding protrusion from an insertion tool.

As shown in FIGS. 7 and 8, the central portion 421 of the second elongate member 420 has a smaller outer diameter than that of the engagement portion 425 of the second elongate member 420 such that the engagement portion 425 includes a shoulder 426. The shoulder 426 is configured to engage a portion of the shoulder 416 of the first elongate member 410. In this manner, movement of the second elongate member 420 distally within the first elongate member 410 is limited by the shoulder 426 of the second elongate member 420 and the shoulder 416 of the first elongate member 410.

The distal end portion 422 of the second elongate member 420 includes an actuation portion 423. As shown in FIGS. 7 and 8, the actuation portion 423 includes a shoulder 451 that contacts a distal end surface 448 of the first elongate member 410. Similarly stated, an outer diameter of the actuation portion 423 is greater than the outer diameter of the central portion 421 of the second elongate member 420 such that the actuation portion 423 forms the shoulder 451. Moreover, the outer diameter of the actuation portion 423 is greater than an inner diameter of the lumen 441 of the first elongate member 410 such that the shoulder 451 contacts the distal end surface 448 of the first elongate member 410. In this manner, as described below, movement of the second elongate member 420 proximally within the first elongate member 410 results in a force being applied to the distal end surface 448 of the first elongate member 410 by the actuation portion 423 of the second elongate member 420.

An outer surface of the distal end portion 422 of the second elongate member 420 includes a rounded or tapered tip. In this manner, the second elongate member 420 is configured to dilate and/or distract bodily tissue. Although shown as being rounded, in some embodiments, for example, the distal end portion 422 of the second elongate member 420 can be configured to pierce bone tissue.

As shown in FIGS. 5-8, the nut 430 includes a threaded portion 431 and an outer surface 432. The threaded portion 431 defines female threads within a lumen of the nut 430 such that the nut 430 can be threadedly coupled to the first elongate member 410. As described above, the threaded portion 432 of the nut 430 corresponds to the threaded portion 412 of the first elongate member 410. The outer surface 432 includes a series of flats (e.g., a hexagonal shape) such that the nut can be received and manipulated by within a portion of an insertion tool (not shown in FIGS. 5-8). In this manner, as described in more detail below, the nut 430 can be rotated relative to the first elongate member 410 such that the nut 430 is moved relative to the first elongate member 410 along the longitudinal axis A_{L1}.

The bone fixation device 400 can be moved between a first configuration (FIGS. 3, 5 and 7) and a second configuration (FIGS. 4, 6 and 8). When the bone fixation device 400 is in the first configuration, the retention portion 414 of the first elongate member 410 has a first size, orientation and/or shape. More particularly, when the bone fixation device 400 is in the first configuration, the collapsible portions 446 are substantially linear and are substantially parallel to (or curved about) the longitudinal axis A_{L1}. Similarly stated, when the bone fixation device 400 is in the first configuration, an outer surface 447 of each collapsible portion 446 is substantially aligned with an outer surface of the central portion 415 of the first elongate member 410. Said another way, the outer surface 447 of each collapsible portion 446 and the outer surface of the central portion 415 form a substantially continuous surface. When viewed in a two-dimensional cross-section (e.g., the cross-section presented in the perspective view of FIG. 7), the outer surface 447 of each collapsible portion 446 is substantially parallel to the outer surface of the central portion 415 of the first elongate member 410.

When the bone fixation device 400 is in the first configuration, the outer surface 447 of each collapsible portion 446 is spaced apart from the longitudinal axis A_{L1} by a first distance. The first distance corresponds to the outer diameter of the retention portion 414 of the first elongate member 410. Similarly stated, when the bone fixation device 400 is in the first configuration, the retention portion 414 has a first size.

When the bone fixation device 400 is in the first configuration, movement of the central portion 421 of the second elongate member 420 within the first elongate member 410 is limited. More particularly, in the first configuration, the shoulder 451 of the actuation portion 423 of the second elongate member 420 contacts the distal end surface 448 of the first elongate member 410, thereby limiting axial movement of the second elongate member 420 within the first elongate member 410 in a proximal direction. Similarly, the shoulder 426 of the second elongate member 420 contacts the shoulder 416 of the first elongate member 410, thus limiting movement of the second elongate member 420 relative to the first elongate member 410 in a distal direction.

As best shown in FIG. 8, the bone fixation device 400 can be moved from the first configuration to the second configuration by moving the second elongate member 420 proximally relative to the first elongate member 410 in a direction shown by the arrow CC. In this manner, the actuation portion 423 of the second elongate member 420 exerts a force on the distal end portion 413 of the first elongate member 410 to move the bone fixation device 400 from the first configuration to the second configuration. More particularly, the insertion tool (not shown) maintains the proximal end portion 411 of the first elongate member 410 in a fixed position and the shoulder 451 of the actuation portion 423 exerts a compressive force on the distal end surface 448 of the first elongate member 410, thereby causing the retention portion 414 to deform. Similarly stated, the actuation portion 423 deforms the collapsible portions 446 when the second elongate member 420 is moved proximally relative to the first elongate member 410. The retention portion 414 can deform plastically, elastically, or a combination thereof.

When the bone fixation device 400 is in the second configuration, the retention portion 414 of the first elongate member 410 has a second size, orientation and/or shape different from the first size, orientation and/or shape described above. More particularly, when the bone fixation device 400 is in the second configuration, the collapsible portions 446 are deformed such that the collapsible portions 446 are non-parallel to the longitudinal axis A_{L1}. Similarly stated, when the bone fixation device 400 is in the second configuration, the outer surface 447 of each collapsible portion 446 is substantially no longer aligned with the outer surface of the central portion 415 of the first elongate member 410. When viewed in a two-dimensional cross-section (see e.g., the cross-section presented in the perspective view shown in FIG. 8), a portion of the outer surface 447 of each collapsible portion 446 is substantially normal to the outer surface of the central portion 415 of the first elongate member 410 and/or the longitudinal axis A_{L1}.

When the bone fixation device 400 is in the second configuration, at least a portion of the outer surface 447 of each collapsible portion 446 is spaced apart from the longitudinal axis A_{L1} by a second distance greater than the first distance. The second distance corresponds to the outer diameter of the retention portion 414 of the first elongate member 410 when the bone fixation device 400 is in the second configuration. Similarly stated, when the bone fixation device 400 is in the first configuration, the retention portion 414 has a second size greater than the first size.

The bone fixation device 400 can be moved from the first configuration to the second configuration, for example, by applying mechanical forces to the first and second elongate members 410 and 420 via an insertion tool (not shown). The insertion tool can be a medical device similar to those disclosed in U.S. patent application Ser. No. 12/112,701, entitled “Apparatus and Methods for Inserting Facet Screws,” filed Apr. 30, 2008, which is incorporated herein by reference in its entirety. Alternatively, the insertion tool can be configured to apply a mechanical force to the second elongate member 420 in the direction CC (see FIG. 14) and further configured to apply a mechanical force to the first elongate member 410 in a direction opposite the direction CC. For example, a first portion of an insertion tool including male threads can be threadedly coupled to the engagement portion 425 of the second elongate member 420 and pulled in the direction CC while a second portion of the insertion tool contacts a proximal end surface of the first elongate member 410, restricting movement of the first elongate member 410 and causing deformation of the retention portion 414. After movement of the bone fixation device 400 from the first configuration to the second configuration, the insertion tool can be disengaged from the bone fixation device 400 by decoupling the tool from the engagement portion 425. Thus, the engagement portion 425 is configured to allow for repeated engagement and disengagement with an insertion tool.

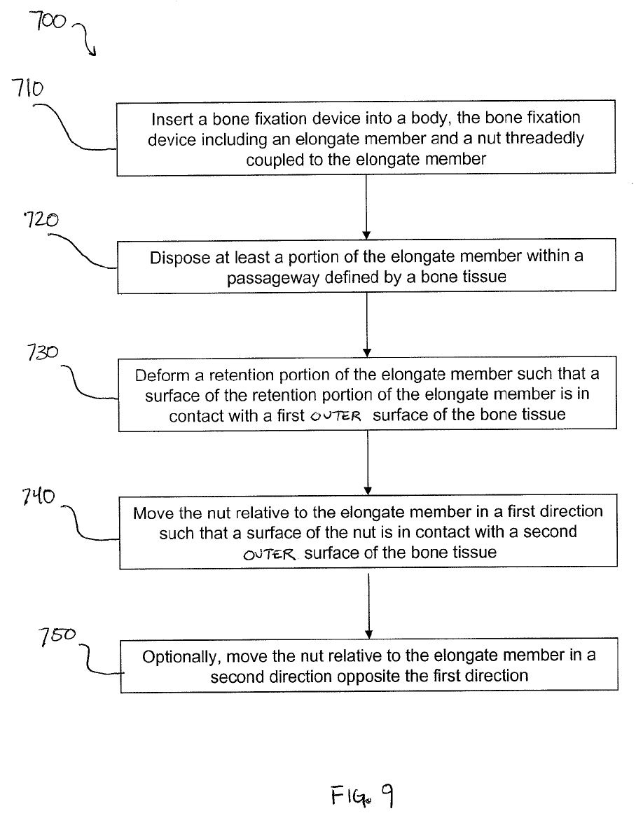

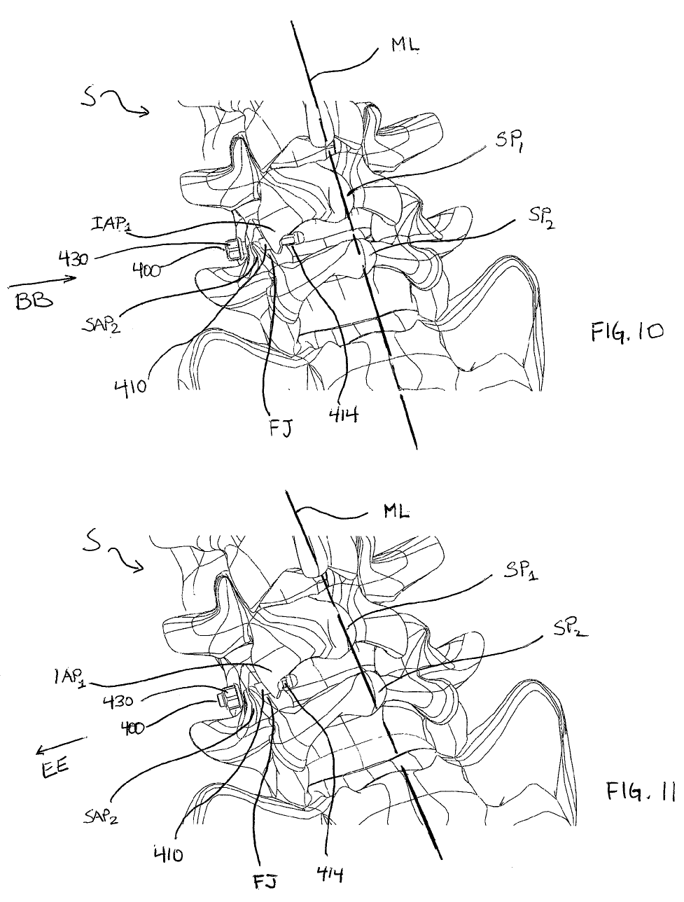

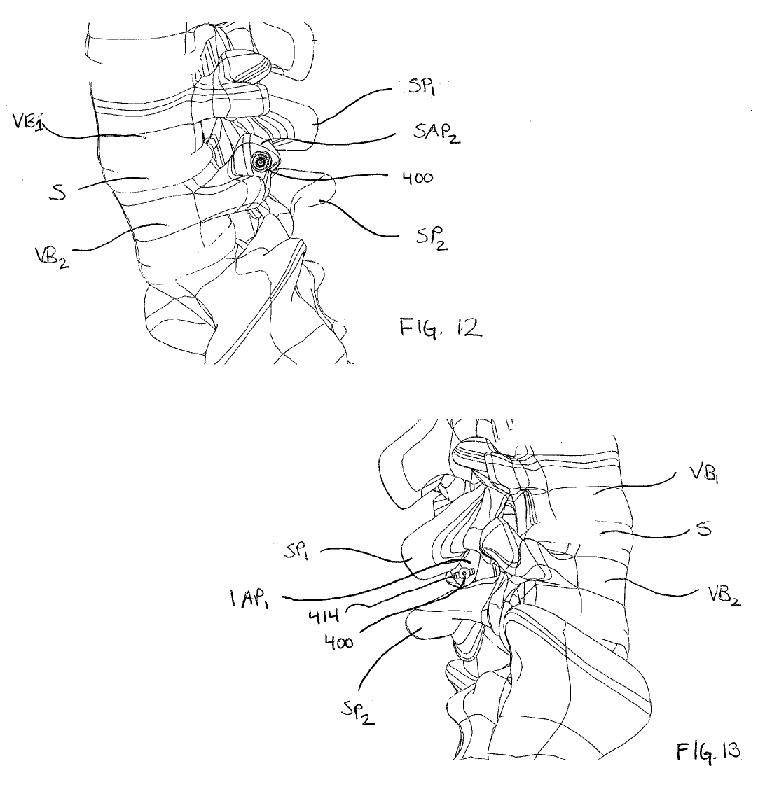

FIG. 9 is a flow chart of a method 700 for disposing a bone fixation device within a body, according to an embodiment. The method illustrated in FIG. 9 is discussed with reference to FIGS. 10-13, which are perspective views of the bone fixation device 400 (as discussed with reference to FIGS. 3-8) disposed within a portion of a spine S in a first configuration (FIGS. 10 and 12) and a second configuration (FIGS. 11 and 13). The spine S has a midline ML axis (see FIGS. 10 and 11), a superior vertebral body VB₁, and an inferior vertebral body VB₂ (see FIGS. 12 and 13). As best shown in FIGS. 10 and 11, the superior vertebral body VB₁ includes a spinous process SP₁ and an inferior articulate process IAP₁. The inferior vertebral body VB₂ includes a spinous process SP₂ and a superior articulate process SAP₂. A region between the inferior articulate process IAP₁ and the superior articulate process SAP₂ defines a facet joint FJ. Although the method 700 is discussed with reference to the bone fixation device 400, the method 700 can be performed with any suitable bone fixation device. Similarly, although the method 700 is discussed with reference to disposing a bone fixation device in a particular bone structure and/or in a particular orientation, in other embodiments, the method 700 can include disposing a bone fixation device in any suitable bone structure and/or in any suitable orientation.

The illustrated method includes inserting a bone fixation device into the body, at 710. Referring to FIGS. 10 and 11, the bone fixation device 400 includes an elongate member (e.g., the first elongate member 410 as shown in FIGS. 3-8) and nut 430 threadedly coupled to the elongate member 410. The bone fixation device 400 can be inserted in any suitable manner. For example, in some embodiments, the bone fixation device 400 can be inserted into the body percutaneously and/or in a minimally-invasive manner. In some embodiments, the bone fixation device 400 can be inserted through a lateral skin incision (i.e., a skin incision offset from the midline axis ML of the spine S). In some embodiments, the lateral skin incision can have a length of between 3 mm and 25 mm. In some embodiments, for example, the lateral skin incision can have a length of approximately 10 mm. Moreover, in some embodiments, the bone fixation device 400 can be inserted into the body via a cannula (not shown in FIGS. 10-13). In some embodiments, such a cannula can have a size of between 3 mm and 25 mm. In some embodiments, for example, the size of the cannula can be approximately 10 mm.

The bone fixation device 400 can be inserted into the body using any suitable insertion tool or tools. For example, in some embodiments, a proximal portion of the bone fixation device 400 can be removably coupled to an insertion tool (not shown in FIGS. 10-13), as described above. The insertion tool can then be used to insert the bone fixation device 400 into the body. In some embodiments, the inserting can include first inserting a guide member (e.g., a guide wire, a Kirschner wire or the like, not shown in FIGS. 10-13) into the body. In this manner, the guide member can pierce, dilate and/or distract bodily tissue to define a passageway within the body. The bone fixation device 400 can then be placed over the guide member and inserted into the body along the predefined passageway. More particularly, the bone fixation device 400 can be disposed about the guide member such that a portion of the guide member is disposed within the lumen 424 of the second elongate member 420 of the bone fixation device 400 (see e.g., FIGS. 7 and 8).

Returning to the flow chart shown in FIG. 9, at least a portion of the elongate member is disposed within a passageway defined within a bone tissue, at 720. Referring to FIGS. 10-13, in some embodiments, the bone fixation device 400 can be moved in a lateral-to-medial direction, as indicated by the arrow BB in FIG. 10. Similarly stated, in some embodiments, the bone fixation device 400 can be moved within the body from a lateral incision (not shown) towards the midline axis ML. Said another way, in the lateral-to-medial approach, the bone fixation device 400 enters the body, distal end first, through an incision made at a first distance from the midline ML of the spine. After insertion, the bone fixation device 400 is moved within the body until the bone fixation device 400 is disposed a second distance from the midline ML, the second distance less than the first distance. In certain instances, the lateral-to-medial approach can prevent and/or minimize interference and/or contact between the bone fixation device 400 and collateral bone structures such as the spinous processes SP₁ and SP₂.

Referring to FIGS. 10-13, in some embodiments, the bone tissue can include more than one bone structure. In some embodiments, for example, the bone tissue can include the superior vertebral body VB1 and the inferior vertebral body VB2, and passageway can be defined within the inferior articulate process IAP₁ and the superior articulate process SAP₂ such that a central portion of the bone fixation device 400 (e.g., the central portion 415 of the first elongate member 410) spans the facet joint FJ. In some embodiments, the bone fixation device 400 can be disposed within the passageway (not shown FIGS. 10-13) such that the central portion 415 of the first elongate member 410 can be disposed within the bone tissue (e.g., the inferior articulate process IAP₁ and/or the superior articulate process SAP₂) while the nut 430 and/or the retention portion 414 are disposed outside of the passageway and/or the bone tissue. In this manner, as described in more detail herein, when the bone fixation device 400 is moved from a first configuration to a second configuration, the nut 430 and/or the retention portion 414 can contact an outer surface of the bone tissue.

Returning to the flow chart shown in FIG. 9, a retention portion of the elongate member of the bone fixation device is deformed such that a surface of the retention portion is in contact with a first outer surface of the bone tissue, at 730. Referring to FIGS. 3-8 and 10-13, the retention portion 414 is deformed such that the surfaces 447 of the collapsible portions 446 contact an outer surface of the inferior articulate process IAP₁ of the superior vertebral body VB₁. In this manner, proximal movement of the bone fixation device 400 within the passageway is limited. The retention portion of the bone fixation device can be deformed in any suitable manner, such as, for example, by moving the second elongate member 420 within the first elongate member 410 in a proximal direction, as shown by the arrow CC in FIGS. 6 and 8, and the arrow EE in FIG. 11.

Returning to the flow chart shown in FIG. 9, a nut is moved relative to the elongate member of the bone fixation device such that a surface of the nut is in contact with a second outer surface of the bone tissue, at 740. Referring to FIGS. 3-8 and 10-13, the nut 430 can be moved relative to the first elongate member 410 until a lower surface of the nut 430 contacts an outer surface of the superior articulate process SAP₂ of the inferior vertebral body VB₂. In this manner, distal movement of the bone fixation device 400 within the passageway is limited. Thus, after the retention portion is deformed (at 730) and the nut is tightened (at 740), movement of the inferior articulate process IAP₁ of the superior vertebral body VB₁ relative to the superior articulate process SAP₂ of the inferior vertebral body VB₂ is limited. Similarly stated, the facet joint FJ is stabilized and/or fixed by the bone fixation device.

The nut 430 can be moved relative to the first elongate member 410 by rotating the nut about the longitudinal axis A_{L1}, as shown by the arrow DD in FIGS. 5 and 6. In some embodiments, the nut 430 can be rotated by the same tool used to insert the bone fixation device and/or dispose the bone fixation device within the bone tissue. In other embodiments, the nut 430 can be rotated by a tool different than the tool used to insert the bone fixation device.

In some embodiments, the nut 430 can be tightened by moving the nut 430 about the longitudinal axis A_{L1} in a first direction (e.g., clockwise). In some embodiments, the illustrated method optionally includes moving the nut relative to the elongate member in a second direction opposite the direction, at 750. Similarly stated, in some embodiments, the method optionally includes loosening the nut. Referring to FIGS. 10-13, in some embodiments, the nut can be loosened until the lower surface of the nut 430 is disposed apart from the outer surface of the superior articulate process SAP₂ of the inferior vertebral body VB₂. In this manner, the bone fixation device 400 can be repositioned within the body. In other embodiments, the nut 430 can be removed from the first elongate member 410. In this manner, the bone fixation device 400 can be removed from the bone tissue.

In some embodiments, the deformation operation 730 and fixation operation 740 are performed independently of one another. For example, in some embodiments, the nut can be moved relative to the elongate member before the retention portion is deformed. In other embodiments, the nut can be moved relative to the elongate member after the retention portion is deformed. In yet other embodiments, the nut can be moved relative to the elongate member and the retention portion can be deformed substantially simultaneously.

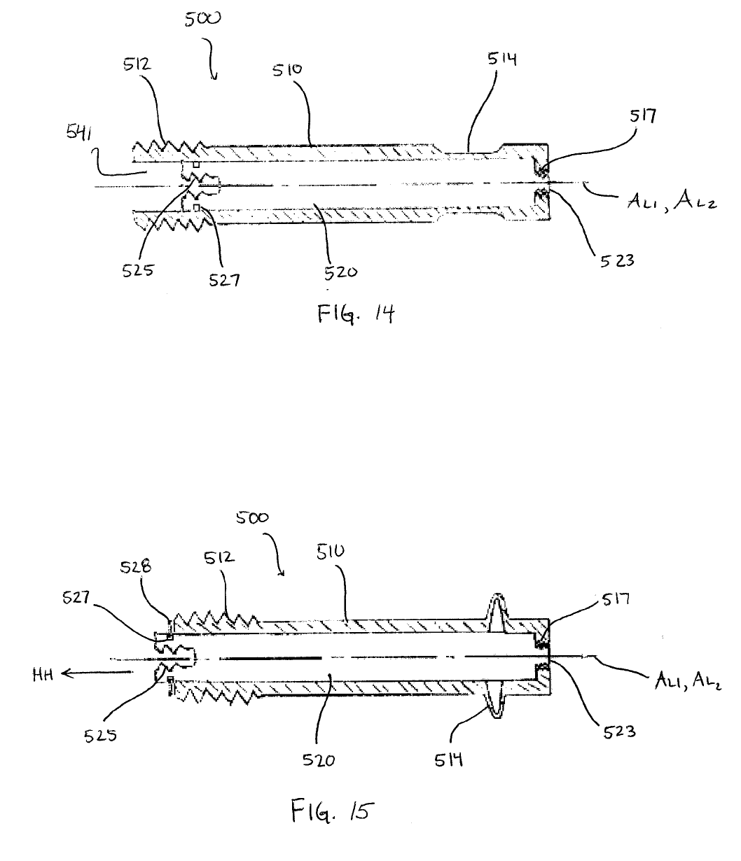

FIGS. 14 and 15 show a bone fixation device 500 according to another embodiment in a first and second configuration, respectively. The device 500 includes a first elongate member 510, a second elongate member 520, and a nut (not shown) configured to threadedly engage the first elongate member 510. The first elongate member 510 defines a longitudinal axis A_{L1} and a lumen 541 that is substantially centered about the longitudinal axis A_{L1}. The first elongate member 510 includes a proximal threaded portion 512, a retention portion 514, and a distal threaded portion 517. The proximal threaded portion 512 of the first elongate member 510 defines male threads configured to engage the female threads of the nut (not shown).

The retention portion 514 includes a side wall having a thickness less than a thickness of adjacent portions of the first elongate member 510. In this manner, the retention portion 514 is configured to deform when a compressive force is applied to the first elongate member 510, as described above. Similarly stated, this arrangement allows the retention portion 514 to deform when the bone fixation device 500 is moved between the first configuration (FIG. 14) and the second configuration (FIG. 15). Although the side wall of the retention portion 514 is shown as having a substantially constant diameter relative to the longitudinal axis A_{L1} and a substantially constant wall thickness, in other embodiments, the side wall of the retention portion 514 can be contoured to extend slightly radially outward from remaining portion of the first elongate member 510. In this manner, the retention portion 514 is biased such that when a compressive force is applied to the first elongate member 510, the retention portion 514 will extend outwardly from the first elongate member 510 (see e.g., FIG. 15). In yet other embodiments, the retention portion 514 can be biased by including a notch in one or more locations along the side wall of the retention portion 514. In yet other embodiments, the retention portion 514 can be biased by varying the thickness of side wall of the retention portion 514 in an axial direction.

The distal threaded portion 517 of the first elongate member 510 defines female threads that are substantially concentric with the longitudinal axis A_{L1}. The distal threaded portion 517 of the first elongate member 510 corresponds to (i.e., has the same nominal size and thread pitch) the threaded portion 523 of the second elongate member 520. In this manner, the second elongate member 520 can be threadedly coupled to the first elongate member 510.

The second elongate member 520 defines a longitudinal axis A_{L2} that is substantially coincident with the longitudinal axis A_{L1}. The second elongate member 520 includes an threaded portion 523, an engagement portion 525, and defines a retaining groove 527. The threaded portion 523 defines male threads configured to engage the distal threaded portion 517 of the first elongate member 510, as described above.

The engagement portion 525 is configured to engage an insertion and/or actuation tool (not shown in FIGS. 14 and 15). More particularly, the engagement portion 525 defines female threads located on a proximal end portion of the second elongate member 520. Thus, an insertion and/or actuation tool can be threadedly coupled to the second elongate member 520 such that movement of the insertion and/or actuation tool along the longitudinal axis A_{L2} results in movement of the second elongate member 520 along the longitudinal axis A_{L2}.

The retaining groove 527 is a region of the proximal end portion of the second elongate member 520 having a minimum outer diameter smaller than the outer diameter of the surrounding proximal end portion. Similarly stated, the retaining groove 527 is a circumferential groove about the proximal end portion of the second elongate member 520. As described in more detail below, the retaining groove is configured to contain a retaining ring 528.

The bone fixation device 500 can be moved between a first configuration (FIG. 14) and a second configuration (FIG. 15). When the bone fixation device 500 is in the first configuration, the entire second elongate member 520 is disposed within the first elongate member 510, and the retention portion 514 of the first elongate member 510 has a first size, orientation and/or shape. More particularly, when the bone fixation device 500 is in the first configuration, at least a portion of the retention portion 514 is substantially parallel to the longitudinal axis A_{L1}. When viewed in a two-dimensional cross-section (as shown in FIG. 14), a surface of the retention portion is substantially parallel to a surface of the remaining portions of the first elongate member 510.

When the bone fixation device 500 is in the first configuration, a portion of the retention portion 514 is spaced apart from the longitudinal axis A_{L1} by a first distance. The first distance corresponds to the outer diameter of the retention portion 514 of the first elongate member 510. Similarly stated, when the bone fixation device 500 is in the first configuration, the retention portion 514 has a first size.

As shown in FIG. 15, the bone fixation device 500 can be moved from the first configuration to the second configuration by moving the second elongate member 520 proximally relative to the first elongate member 510 in a direction shown by the arrow HH. Because the threaded portion 523 of the second elongate member 520 is threadedly engaged to the distal threaded portion 517 of the first elongate member 510, the relative movement of the second elongate member 520 within the first elongate member 510 produces a force on the first elongate member 510. Thus, the retention portion 514 of the first elongate member 510 can be deformed.

When the bone fixation device 500 is in the second configuration, a proximal end portion of the second elongate member 520 is disposed outside the first elongate member 510, exposing the retaining groove 527. A retaining ring 528 can then be placed in the groove 527, the retaining ring 528 being configured to contact both a surface of the groove 527 and a distal end surface of the first elongate member 510 to prevent subsequent movement of the bone fixation device 500 back to the first configuration. For example, in some embodiments, the retention portion 514 of the first elongate member 510 is elastically deformed when the bone fixation device 500 is moved from the first configuration to the second configuration. In such embodiments, the retaining ring 528 can prevent the elastic forces from moving the bone fixation device 500 from the second configuration back to the first configuration. In other embodiments, the retention portion 514 of the first elongate member 510 is plastically deformed when the bone fixation device 500 is moved from the first configuration to the second configuration. In such embodiments, the retaining ring 528 can prevent the retention portion 514 from creeping from the second configuration back to the first configuration. The retaining ring 528, for example, can be included within an insertion tool and can be placed within the retaining groove 527 by the insertion tool.

In yet other embodiments, the retaining groove 527 need not be present, and the second elongate member 520 can be decoupled and removed entirely from the first elongate member 510 after deformation of the retention portion 514. The second elongate member 520 can be decoupled, for example, by rotating the second elongate member 520 relative to the first elongate member 510 to disengage the distal threaded portion 517 from the threaded portion 523 of the second elongate member 520. In some embodiments, the second elongate member 520 can thus be reusable for multiple procedures using a new first elongate member 510 for each procedure. In some embodiments, the second elongate member 520 can be monolithically constructed with a reusable insertion tool and thus engagement portion 525 need not be present.

As shown in FIG. 15, when the bone fixation device 500 is in the second configuration, the retention portion 514 of the first elongate member 510 has a second size, orientation and/or shape different from the first size, orientation and/or shape described above. More particularly, when the bone fixation device 500 is in the second configuration, the retention portion 514 is deformed such that a surface of the retention portion 514 is non-parallel to the longitudinal axis A_{L1}. More particularly, when viewed in a two-dimensional cross-section, at least a portion of a surface of the retention portion 514 is substantially normal to the outer surface of the first elongate member 510 and/or the longitudinal axis A_{L1}.

When the bone fixation device 500 is in the second configuration, a portion of the retention portion 514 is spaced apart from the longitudinal axis A_{L1} by a second distance greater than the first distance. The second distance corresponds to the outer diameter of the retention portion 514 of the first elongate member 510 when the bone fixation device 500 is in the second configuration. Similarly stated, when the bone fixation device 500 is in the first configuration, the retention portion 514 has a second size greater than the first size.

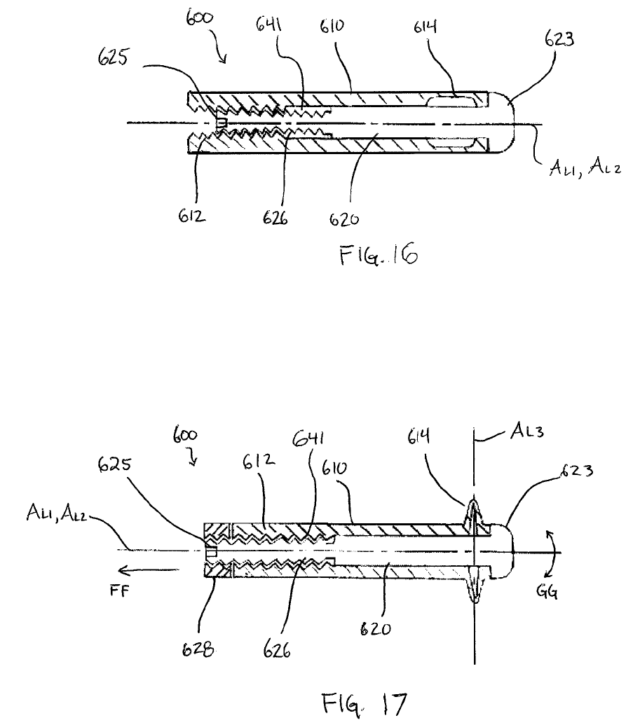

FIGS. 16 and 17 show a bone fixation device 600 according to another embodiment in a first configuration and a second configuration, respectively. The bone fixation device 600 has a first elongate member 610 a second elongate member 620. The first elongate member 610 defines a longitudinal axis A_{L1} and a lumen 641 that is substantially concentric to the longitudinal axis A_{L1}. The first elongate member 610 includes a threaded portion 612 and a retention portion 614. The threaded portion 612 includes female threads at a proximal end portion of the first elongate member 610. The retention portion 614 includes a side wall having a thickness less than a thickness of adjacent regions of the first elongate member 610. As described above, this geometry allows the retention portion 614 to deform when a compressive force is applied to the first elongate member 610.

The second elongate member 620 defines a longitudinal axis A_{L2} that is substantially coincident with the longitudinal axis A_{L1}. The second elongate member 620 includes an actuation portion 623, a threaded portion 626, and an engagement portion 625. The actuation portion 623 is in contact with a distal end surface of the first elongate member 610 and is configured to move freely on that surface when the second elongate member 620 is rotated about the longitudinal axis L_{A2}, as shown by the arrow GG in FIG. 17. The threaded portion 626 defines male threads at a proximal end portion of the second elongate member 620. The male threads of the threaded portion 626 correspond to (i.e., has the same nominal size and thread pitch) female threads of the threaded portion 612. In this manner, the threaded portion 626 of the first elongate member 610 is threadedly coupled to the threaded portion 612 of the first elongate member 610. The engagement portion 625 defines a hexagonal socket configured to receive an insertion tool (not shown).

The bone fixation device 600 can be moved between a first configuration (FIG. 16) and a second configuration (FIG. 17). When the bone fixation device 600 is in the first configuration, an outer surface of the side wall of the retention portion 614 is substantially aligned with an outer surface of the adjacent portions of the first elongate member 610. An inner surface of the side wall of the retention portion 614 is recessed from an inner surface of the adjacent portions of the first elongate member 610. When viewed in a two-dimensional cross-section (i.e., as shown in FIGS. 16), an external surface of the retention portion 614 is substantially parallel to the longitudinal axis A_{L1}.

An insertion tool configured with a hexagonal driver can be rotationally coupled to the engagement portion 625 of the second elongate member 620. Similarly stated, a portion of an insertion tool can be disposed within the hexagonal socket of the engagement portion 625 such that rotation of the insertion tool results in rotation of the second elongate member 620. The insertion tool can optionally be further configured to restrict movement of the first elongate member 610 by contacting a proximal end surface thereof. In some embodiments, for example, an insertion tool can be configured to limit the rotation of the first elongate member such that the second elongate member 620 can be rotated within (and relative to) the first elongate member 610. Such insertion tools can be, for example, any of the tools shown and described in U.S. patent application Ser. No. 12/112,701, entitled “Apparatus and Methods for Inserting Facet Screws,” filed Apr. 30, 2008, which is incorporated herein by reference in its entirety.

Rotation of the coupled insertion tool and second elongate member 620 about the longitudinal axis A_{L2}, as shown by arrows GG, thus moves the second elongate member 620 relative to the first elongate member 610 in a proximal direction FF parallel to the longitudinal axis A_{L2}. The proximal movement of the second elongate member causes the actuation portion 623 of the second elongate member 620 to deform the retention portion 614. In this manner, or by other suitable means, the bone fixation device 600 can be moved to one or more different configurations (e.g., the second configuration, as shown in FIG. 17).

In the second configuration, the retention portion 614 is deformed such that portions of the external surface of the retention portion 614 are substantially parallel to the longitudinal axis A_{L1} and substantially perpendicular to an external surface of a different portion of the first elongate member 610. In this manner, as described above, the retention portion 614 can limit axial movement of the bone fixation device 600 when the bone fixation device 600 is disposed within bone tissue. As shown in FIG. 17, a retaining nut 628 locks the bone fixation device 600 in the second configuration by engaging the threaded portion 626 of the second elongate member and a proximal end surface of the first elongate member 610. As shown in FIG. 17, the retaining nut 628 has a smaller outer diameter than a central portion of the first elongate member 610 to minimize the overall profile of the bone fixation device 600 during insertion into the body. The overall profile of the insertion tool can likewise be reduced if the retaining nut 628 is a castellated nut. The retaining nut can be, for example, a castellated nut of the types shown and described in U.S. patent application Ser. No. 12/112,701, entitled “Apparatus and Methods for Inserting Facet Screws,” filed Apr. 30, 2008, which is incorporated herein by reference in its entirety. A smaller bone fixation device profile or a smaller insertion tool profile yields the advantage of a smaller body incision and a generally less invasive medical procedure. This approach for reducing the size of the bone fixation device can be applied in other embodiments as well, such as, for example, by defining the nut 430 of the bone fixation device 400 as a castellated nut.

FIG. 18 is a cross-sectional view of a bone fixation device 600′ in a second configuration, according to another embodiment. The bone fixation device 600′ is similar to the bone fixation device 600 shown and described above, and is therefore not described in detail. The bone fixation device 600′ differs from the bone fixation device 600, however, in that the outer diameter of the retaining nut 628′ is larger than the outer diameter of the first elongate member 610. In this manner, when the bone fixation device 600′ is in the second configuration (as shown in FIG. 18), a surface of the retaining nut 628 is configured to engage an external surface of a bone tissue. If the bone fixation device 600 is configured as shown in FIG. 18, the desired distance between the retention portion 614 and the retaining nut 628 can be determined to substantially correspond to (e.g., to fit within) the size of the bone tissue in which the bone fixation device 600 will be disposed.

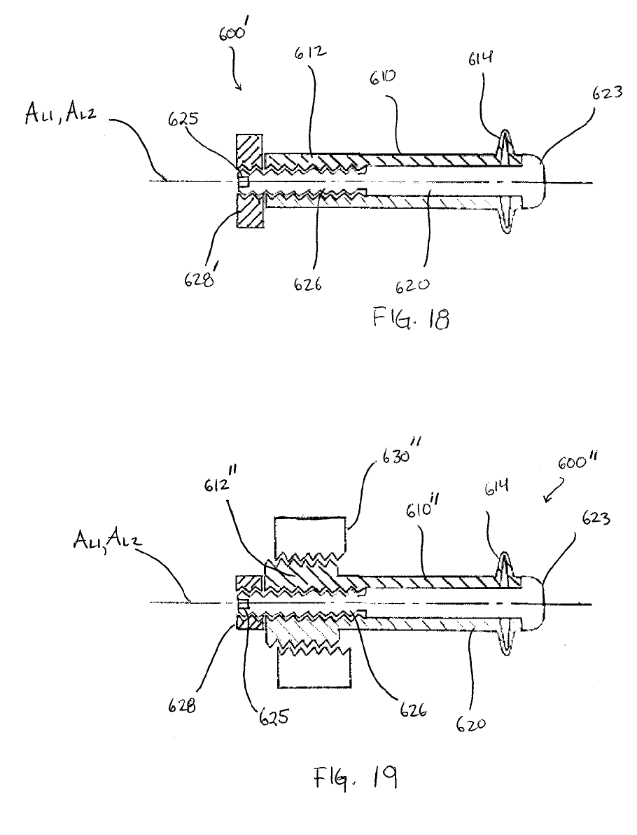

FIG. 19 is a cross-sectional view of a bone fixation device 600″ in a second configuration, according to another embodiment. The bone fixation device 600″ is similar to the bone fixation device 600 shown and described above, and is therefore not described in detail. The bone fixation device 600″ differs from the bone fixation device 600, however, in that the threaded portion 612″ of the first elongate member 610″ also includes male threads disposed at proximal end portion of the first elongate member 610″. The male threads are configured to engage the female threads of a nut 630″. Thus, when the bone fixation device 600″ is in the second configuration (as shown in FIG. 19), the nut 630″ can be threadedly disposed about the first elongate member 610″. In this manner, movement of the bone fixation device 600″ within a bone tissue can be limited.

Although various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above. Thus, the breadth and scope of the invention should not be limited by any of the above-described embodiments. While the invention has been particularly shown and described with reference to specific embodiments thereof, it will be understood that various changes in form and details may be made.

For example, although the bone fixation devices are shown and described above as including a actuator (e.g., actuation portion 123, actuation portion 423 or the like) configured to move a retention portion (e.g., retention portion 114, retention portion 424 or the like) between a first configuration and a second configuration, in other embodiments, a bone fixation device need not include an actuator and/or an actuation portion. For example, in some embodiments, a bone fixation device can include a retention portion configured to move between a first configuration and a second configuration without being externally actuated. For example, in some embodiments, a retention portion can be constructed from a shape-memory alloy, such as, for example Nitinol, having a first shape when at a first temperature and a second shape when the retention portion is above a transition temperature. The transition temperature can be, for example, slightly below the body temperature such that when the temperature of the retention portion increases upon being inserted into the body, the retention portion changes from its first configuration to its second configuration.

Although the engagement portion 425 of the second elongate member 420 is shown as being configured to be repeatedly and/or reversibly coupled to an insertion tool (e.g., by a threaded coupling), in other embodiments, an engagement portion can be configured to be irreversibly coupled to an insertion tool. Similarly stated, in other embodiments, an engagement portion can be configured to be coupled to an insertion tool only one time. For example, in some embodiments, an engagement portion can include a rod that is fixedly coupled to the insertion tool. The rod can be used to transmit an axial force to an actuation portion of a second elongate member to move a retention portion from a first configuration to a second configuration, as described above. The rod can also be configured to break when exposed to a torsional load above a predetermined value, thereby permanently decoupling the second elongate member from the insertion tool. In this manner, after the axial force has been transmitted to the actuation portion, a user can twist a the rod via the insertion tool, thereby causing the rod to break. In some embodiments, the rod can include grooves, drillings and/or other stress concentration risers to ensure that the rod breaks in a predetermined location and/or when exposed to a torque above a predetermined value.

includes an engagement portion 425 (best shown in FIGS. 7 and 8) configured to engage an insertion tool (not shown in FIGS. 3-8). The engagement portion 425 defines female threads disposed within the lumen 424 such that an insertion tool can be threadedly coupled to the second elongate member 420. In other embodiments, the engagement portion 425 can include any suitable feature for engaging an insertion tool. For example, in some embodiments, the engagement portion 425 can include male threaded portion to be disposed within a corresponding female threaded portion from an insertion tool. In other embodiments, the engagement portion 425 can include a hexagonal shaped recess configured to receive a corresponding protrusion from an insertion tool.

Although the collapsible portions 446 of the retention portion 414 are shown above as being monolithically constructed with the retention portion 414 and/or the distal end portion 413 of the first elongate member 410, in other embodiments, the collapsible portions 446 can be formed separately from and later attached to the retention portion 414 and/or the distal end portion 413 of the first elongate member 410. For example, in some embodiments, the distal end portion 413 of the first elongate member 410 can be constructed from a material having a higher yield strength and/or a higher modulus of elasticity, while the collapsible portions 446 can be constructed from a material having a lower yield strength and/or a lower modulus of elasticity.

Similarly, although the threaded portion 412 of the first elongate member 410 is shown as being monolithically constructed with the retention portion 414 of the first elongate member 410, in other embodiments, the threaded portion 412 of the first elongate member 410 can be formed separately from the retention portion 414 of the first elongate member 410. For example, in some embodiments, a threaded portion can be formed as a sleeve that is disposed about a proximal end portion of a first elongate member. Such a sleeve can be coupled to the proximal end portion by any suitable mechanism, such as, for example, a weld, an interference fit, an epoxy or the like.

Although the bone fixation device 400 is shown and described above as including a nut 430 configured to removably coupled to the first elongate member 410 (i.e., the nut 430 can be loosened and/or removed), in other embodiments, a bone fixation device can include a nut configured to be substantially irreversibly tightened about a portion of a bone fixation device. For example, in some embodiments, the threaded portion 412 of the first elongate member 410 can have a different nominal size and/or thread pitch than the threaded portion 431 of the nut 430. In this manner, after the nut 430 is tightened about the first elongate member 410, the interference between the threaded portion 431 of the nut 430 and the threaded portion 412 of the first elongate member 410 can limit the movement of the nut 430 relative to the first elongate member 410, thereby preventing the nut 430 from being loosened. In other embodiments, the threaded portion 412 of the first elongate member 410 can include a protrusion to limit the movement of the nut 430 relative to the first elongate member 410.

Although the bone fixation device 400 is shown and described above as including a nut 430 configured to be tightened on the first elongate member 410 of the bone fixation device 400, in other embodiments, a bone fixation device can include any suitable device for limiting movement of the bone fixation device and/or applying a clamping load to a bone tissue. For example, in some embodiments, a bone fixation device can include a proximal retention portion configured to limit axial movement of the bone fixation device within a bone tissue. The proximal retention portion can include, for example, one or more deformable portions similar to the deformable portions 446 of the retention portion 414. In this manner, the proximal retention portion can be actuated and/or deformed by applying a compressive force about the proximal portion of the device.

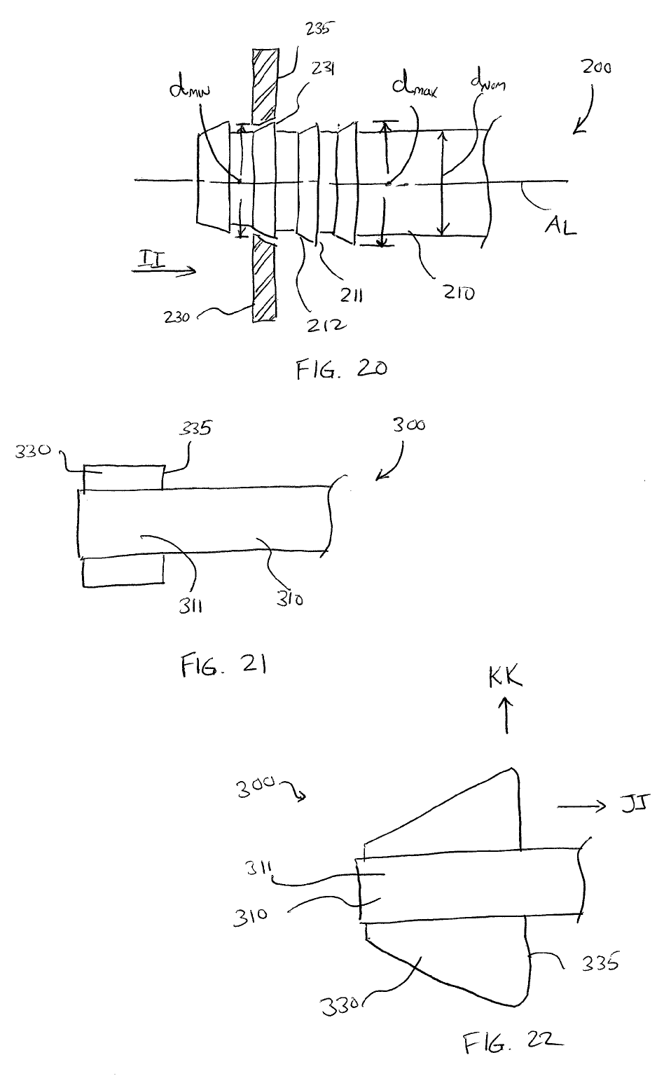

In other embodiments, a bone fixation device can include a coupling member configured to be moved about a portion of the bone fixation device in a non-threaded manner. FIG. 20 is a schematic illustration of a portion of a bone fixation device 200 according to an embodiment. The bone fixation device 200 is similar to the bone fixation devices shown and described above, and is therefore not described in detail. The bone fixation device 200 includes an elongate member 210 and a retention member 230. The elongate member 210 can be similar to the first elongate member 410 shown and described above, and can include, for example, a retention portion (not shown in FIG. 20) having one or more deformable portions. A proximal end portion 211 of the elongate member includes a series of protrusions 212 that are tapered such that the outer diameter of each protrusion 212 decreases along a longitudinal axis AL of the first elongate member in a proximal direction. More particularly, the outer diameter of each protrusion 212 decreases from the maximum diameter dₘₐₓ to nominal diameter dₙₒₘ.

The retention member 230 includes a bone engagement surface 235 and defines a tapered opening 231. The tapered opening 231 corresponds the tapered protrusions 212. The minimum diameter dₘᵢₙ of the tapered opening 231 is smaller than the nominal diameter dₙₒₘ of the elongate member 210. When the retention member 230 is moved distally about the elongate member 210, as shown by the arrow II, the tapered opening 231 can move relative to the tapered protrusions 212. The arrangement of the tapered protrusions 212 and the tapered opening 231, however, prevents the retention member 230 from moving proximally relative to the elongate member 210.

FIGS. 21 and 22 are schematic illustrations of a portion of a bone fixation device 300 according to an embodiment, in a first configuration and a second configuration, respectively. The bone fixation device 300 is similar to the bone fixation devices shown and described above, and is therefore not described in detail. The bone fixation device 300 includes an elongate member 310 and an expandable member 330. The elongate member 310 can be similar to the first elongate member 410 shown and described above, and can include, for example, a retention portion (not shown in FIG. 21) having one or more deformable portions.

The expandable member 330 is coupled to a proximal end portion 311 of the elongate member 310, and includes a bone engagement surface 335. The expandable member 330 is configured to be moved between a first configuration (FIG. 21) and a second configuration (FIG. 22). The expandable member can be, for example, a medical balloon of configured to be inflated with a fluid, such as a gas, saline solution or the like.

In use, the bone fixation device 300 can be inserted into a bone tissue, and a distal end portion (not shown) of the bone fixation device 300 can be expanded to limit movement of the bone fixation device 300 within bone tissue, as described above. The expandable member 330 can then be expanded to limit movement of the bone fixation device and/or applying a clamping load to a bone tissue. More particularly, the expandable member 330 can be expanded such that the bone engagement surface 335 moves distally, as shown by the arrow JJ in FIG. 22. In some embodiments, a portion of the expandable member 330 can expand in a radial direction, as shown by the arrow KK, to provide a greater surface area of contact between the bone engagement surface 335 and the bone tissue.

Although the expandable member 330 is described above as being a medical balloon, in other embodiments, an expandable member can be expanded by means other than being inflated with a fluid. For example, in some embodiments, an expandable member can be expanded by a spring, an elastic member or the like.

Although the actuation portion 423 of the second elongate member 420 is shown as being monolithically constructed with the tool engagement portion 425 of the second elongate member 420, in other embodiments, the actuation portion 423 can be formed separately from the remainder of the second elongate member 420. For example, in some embodiments, the bone fixation device 400 can be assembled by disposing the first elongate member 610 about a portion of the second elongate member 620, excluding the actuation portion 623. The actuation portion 623 can then be coupled to the distal end portion of the second elongate member 620. The actuation portion 623 can be coupled to the distal end portion of the second elongate member 620 in any suitable manner, such as, for example, by welding, by a thermal bond, by an epoxy, or the like.

Although the bone fixation devices are shown and described herein as being disposed and/or deployed within a bone tissue that includes multiple bone structures (e.g., multiple vertebrae), in other embodiments, the bone fixation devices shown and described herein can be disposed and/or deployed within a bone tissue including a single bone structure, such as, for example, a long bone.

Although the bone fixation device 400 is shown as including a second elongate member 420 having at least a portion configured to remain disposed within a first elongate member 410 after the retention portion 414 has been moved from the first configuration to the second configuration, in other embodiments, a bone fixation device can include a second elongate member and/or an actuator configured to be removed from the bone fixation device after the retention portion has been moved from a first configuration to a second configuration. For example, FIGS. 23-26 show a bone fixation device 800 having a removable second elongate member 820 according to an embodiment. The bone fixation device 800 is similar to the bone fixation device 400 shown and described above, and is therefore not described in detail. The bone fixation device 800 differs from the bone fixation device 400, however, in that the second elongate member 820 can be removed from the bone fixation device after the bone fixation device 800 has been deployed within the body.

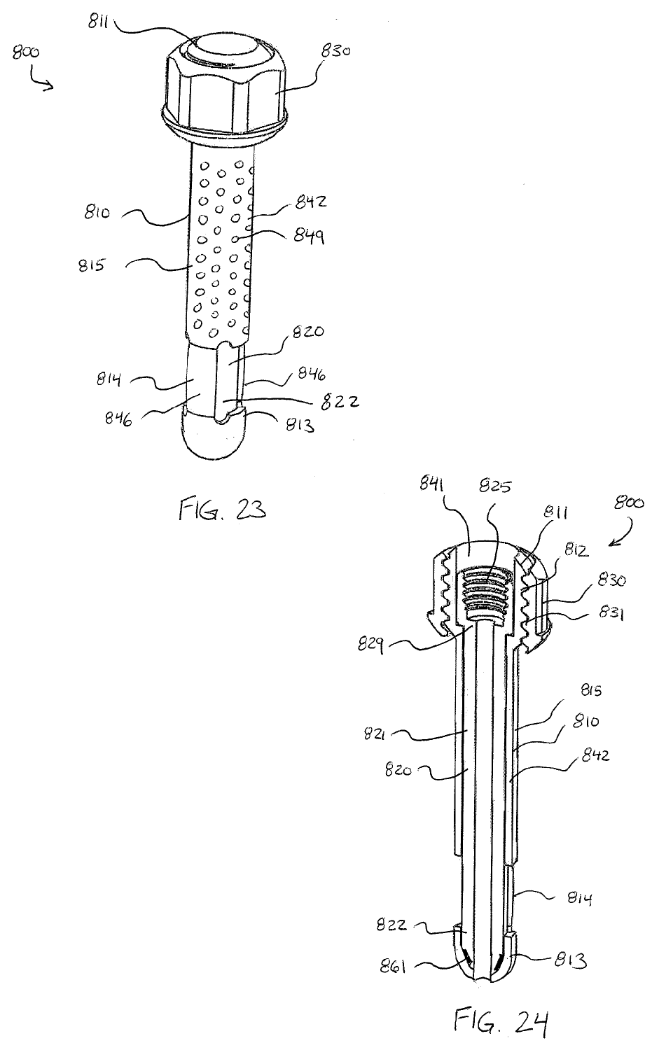

The bone fixation device 800 includes a first elongate member 810, a second elongate member 820, and a nut 830 threadedly coupled to the first elongate member 810. The first elongate member 810 includes a proximal end portion 811, a distal end portion 813, and a central portion 815 between the proximal end portion 811 and the distal end portion 813. The first elongate member 810 includes a side wall 842 that defines a lumen 841 (see FIG. 24). The central portion 815 of side wall 842 also defines a series of openings 849. As described in more detail herein, the openings 849 are configured to allow a bone graft material to be conveyed from the lumen 841 to a region outside of the lumen 841 in a controlled manner to promote fusion of a bone structure. Although the openings 849 are shown as having a circular shape, in other embodiments, the openings 849 can be of any suitable shape and/or size, such as for example, perforations, elongated slots, or the like.

The proximal end portion 811 of the first elongate member 810 includes a threaded portion 812, which is similar to the threaded portion 412 of the bone fixation device 400 as described above. The threaded portion 812 of the first elongate member 810 corresponds to the threaded portion 831 of the nut 830. In this manner, the nut 830 can be threadedly coupled to the first elongate member 810. The distal end portion 813 of the first elongate member 810 includes a retention portion 814, which is similar to the retention portion 414 of the bone fixation device 400 as described above. More particularly, the retention portion 814 defines a pair of collapsible portions 846.

As shown in FIG. 24, the second elongate member 820 includes a proximal end portion 829, a distal end portion 822, and a central portion 821 between the proximal end portion 829 and the distal end portion 822. At least the central portion 821 of the second elongate member 820 and the distal end portion 822 of the second elongate member 820 are disposed within the lumen 841 of the first elongate member 810. The proximal end portion 829 of the second elongate member 820 includes an threaded engagement portion 825 configured to removably engage an insertion tool (not shown in FIGS. 23-26), as described above with reference to the engagement portion 425 shown in FIGS. 3-8.

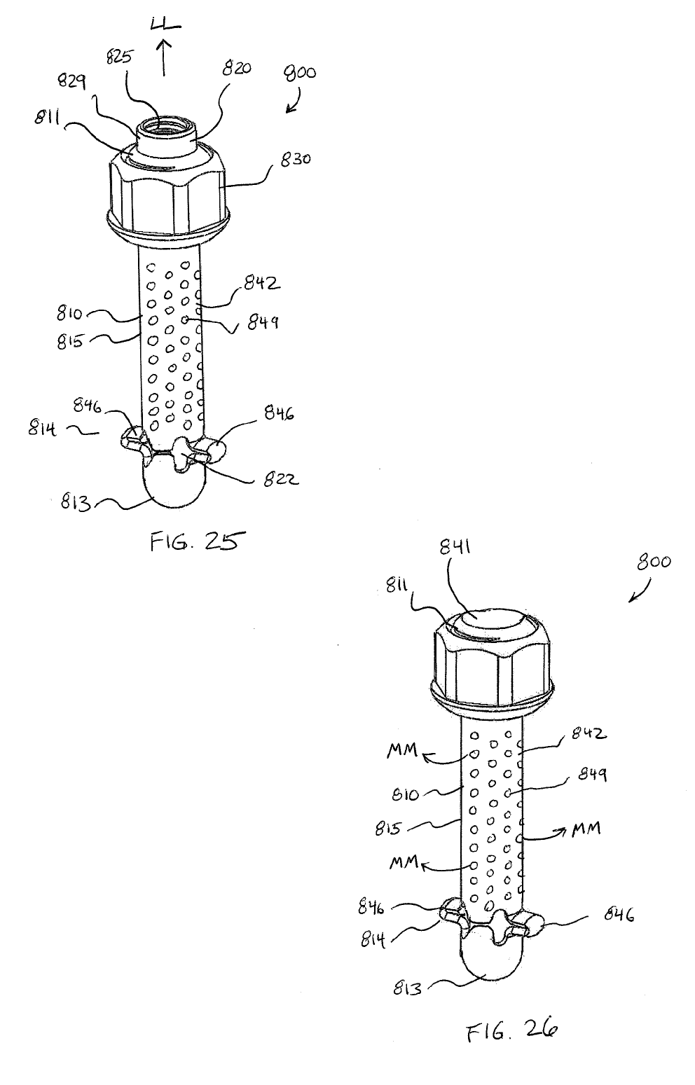

The distal end portion 822 of the second elongate member 820 includes an coupling portion 861 (see FIG. 25) configured to removably couple the second elongate member 820 within the first elongate member 810. More particularly, the coupling portion 861 is configured maintain the coupling between distal end portion 822 of the second elongate member 820 and the distal end portion 813 of the first elongate member 810 when a longitudinal force is exerted on the distal end portion 813 of the first elongate member 810 via the second elongate member 820. Said another way, the coupling portion 861 is configured to prevent the second elongate member 820 from moving relative to the first elongate member 810 when a longitudinal force is exerted on the distal end portion 813 of the first elongate member 810 via the second elongate member 820. In this manner, as described below, movement of the second elongate member 820 proximally within the first elongate member 810 results in a force being applied to the distal end portion 813 of the first elongate member 810 by the coupling portion 861 of the second elongate member 820. Thus, as described in more detail herein, the retention portion 814 of the first elongate member 810 can be deformed from a first configuration to a second configuration via a compressive force applied by the second elongate member 820. Although the coupling portion 861 is shown and described as being a portion of the second elongate member 820, in other embodiments, a coupling portion can be a portion of the first elongate member 810. In yet other embodiments, a coupling portion can be a portion of the first elongate member 810 and the second elongate member 820.

The coupling portion 861 of the second elongate member 820 is further configured to allow the second elongate member 820 to be removed from the first elongate member 810. For example, as described below, in some embodiments, the second elongate member 820 can be removed from the first elongate member 810 after the retention portion 814 has been moved from its first configuration to its second configuration. The coupling portion 861 can include any suitable mechanism for removably coupling the second elongate member 820 within the first elongate member 810. In some embodiments, for example, the coupling portion 861 can include a threaded portion configured to matingly engage a corresponding threaded portion of the first elongate member 810. In other embodiments, the coupling portion 861 can include a weld joint, a solder joint, bond, epoxy or the like configured to maintain the coupling between the second elongate member 820 and the first elongate member 810 when exposed to a longitudinal force, and release the coupling between the second elongate member 820 and the first elongate member 810 when exposed to a torque above a predetermined value. In this manner, after a longitudinal force has been transmitted to the first elongate member 810 via the second elongate member 820, a user can twist a the second elongate member 820 within the first elongate member 810 to decouple the second elongate member 820 from the first elongate member 810.

The bone fixation device 800 can be moved from a first configuration (FIGS. 23 and 24) to a second configuration (FIG. 25) and then to a third configuration (FIG. 26). When the bone fixation device 800 is in the first configuration, the retention portion 814 of the first elongate member 810 has a first size, orientation and/or shape, similar to the size, orientation and/or shape of the retention portion 414 as described above. When the bone fixation device 800 is in the first configuration, longitudinal movement of the central portion 821 of the second elongate member 820 within the first elongate member 810 is limited. More particularly, in the first configuration, the coupling portion 861 of the second elongate member 820 maintains the coupling between distal end portion 822 of the second elongate member 820 and the distal end portion 813 of the first elongate member 810.

As best shown in FIG. 25, the bone fixation device 800 can be moved from the first configuration to the second configuration by moving the second elongate member 820 proximally relative to the first elongate member 810 in a direction shown by the arrow LL in FIG. 25. In this manner, the second elongate member 820 exerts a force on the distal end portion 813 of the first elongate member 810 to move the bone fixation device 800 from the first configuration to the second configuration. More particularly, an insertion tool (not shown) can maintain the proximal end portion 811 of the first elongate member 810 in a fixed position when the second elongate member 820 exerts a force exerts a compressive force on the distal end portion 813 of the first elongate member 810, thereby causing the retention portion 814 to deform. Similarly stated, the collapsible portions 846 of the first elongate member 810 are deformed when the second elongate member 820 is moved proximally relative to the first elongate member 810.

When the bone fixation device 800 is in the second configuration, the retention portion 814 of the first elongate member 810 has a second size, orientation and/or shape different from the first size, orientation and/or shape. More particularly, when the bone fixation device 800 is in the second configuration, the collapsible portions 846 are deformed such that the collapsible portions 846 are non-parallel to a longitudinal axis of the first elongate member 810. Moreover, when the bone fixation device 800 is in the second configuration, the second elongate member 820 remains coupled within the first elongate member 810 by the coupling portion 861.

As best shown in FIG. 26, the bone fixation device 800 can be moved from the second configuration to the third configuration by removing the second elongate member 820 from the first elongate member 810, and by filling the lumen 841 of the first elongate member 810 with a bone graft material. The second elongate member 820 can be removed from the first elongate member 810 by releasing the coupling between the second elongate member 820 (at the coupling portion 861) and the first elongate member 810, and moving second elongate member 820 proximally relative to the first elongate member 810. The coupling between the second elongate member 820 and the first elongate member 810 can be either reversibly released (i.e., released in a manner such that the second elongate member 820 can be re-coupled to the first elongate member 810) or irreversibly released (i.e., released in a manner such that the second elongate member 820 cannot be recoupled to the first elongate member 810). For example, in some embodiments, the second elongate member 820 can be decoupled from the first elongate member 810 by unscrewing a threaded coupling at the coupling portion 861 of the second elongate member 820. In other embodiments, the second elongate member 820 can be decoupled from the first elongate member 810 by irreversibly breaking the coupling portion 861 (e.g., breaking a bond, weld, solder joint or the like).

After the second elongate member 820 is removed from the first elongate member 810, a bone graft material 890 can be conveyed into the lumen 841 of the first elongate member 810. The nut 830 can then be tightened, as described above with respect to the nut 430, to stabilize and/or fix the bone structure. In this manner, the bone fixation device 800 includes a substantially hollow fixation device (e.g., the first elongate member 810) that is filled with bone graft material. When the bone fixation device 800 is in the third configuration, the bone graft material can contact the bone structure via the series of holes 849. Similarly stated, when the bone fixation device 800 is in the third configuration, the bone graft material can be conveyed from the lumen 841 of the first elongate member 810 into contact with the bone structure via the series of holes 849, as shown by the arrows MM in FIG. 26. Thus, when the bone fixation device 800 is in the third configuration, the bone graft material can interdigitate with the bone structure via the holes 849, thereby promoting fusion of the bone structure.

The bone graft material can be any suitable bone graft material configured to promote fusion of the bone structure adjacent the bone fixation device 800. For example, in some embodiments, the bone graft material can include allograft, autograph, or bone morphogenetic proteins (BMPs). The size, shape and/or consistency of the bone graft material can be selected such that the bone graft material can be conveyed from the lumen 841 of the first elongate member 810 via the series of holes 849 in a controlled manner. Similarly stated, the holes 849 can have any suitable size and/or shape such that the bone graft material can be conveyed from the lumen 841 of the first elongate member 810 via the series of holes 849 in a controlled manner. In this manner, the bone graft material can be conveyed from the lumen 841 of the first elongate member 810 over a predetermined time period. In some embodiments, for example, the holes can be circular and can have a diameter of less than 1 millimeter.

Although various embodiments have been described as having particular features and/or combinations of components, other embodiments are possible having a combination of any features and/or components from any of embodiments as discussed above. For example, in some embodiments, a bone fixation device can include collapsible portions similar to those of the bone fixation device 400, a threaded distal end actuation portion similar to that of the bone fixation device 500, and a retaining nut similar to that of the bone fixation device 600. Furthermore, any of the various embodiments and applications of method 700 may employ any of the various embodiments of the bone fixation device disclosed herein.

What is claimed is:

1. An apparatus, comprising:
a first elongate member, a proximal end portion of the first elongate member including a threaded portion, a distal end portion of the first elongate member including a retention portion, the retention portion configured to deform when moved from a first configuration to a second configuration; and
a second elongate member, at least a central portion of the second elongate member being disposed within the first elongate member, a distal end portion of the second elongate member including an actuation portion configured to deform the retention portion of the first elongate member from the first configuration to the second configuration.

2. The apparatus of claim 1, wherein the threaded portion of the first elongate member and the retention portion of the first elongate member are monolithically constructed.

3. The apparatus of claim 1, wherein the threaded portion includes a plurality of external threads, the apparatus further comprising:
a nut configured to engage the plurality of external threads.

4. The apparatus of claim 1, wherein:
the threaded portion of the first elongate member includes a plurality of internal threads; and
a proximal end portion of the second elongate member includes a threaded portion configured to engage the internal threads of the first elongate member.

5. The apparatus of claim 1, wherein a surface of the retention portion of the first elongate member is disposed apart from a longitudinal axis of the first elongate member by a first distance when in the first configuration, the surface of the retention portion of the first elongate member is disposed apart from the longitudinal axis of the first elongate member by a second distance when in the second configuration, the second distance greater than the first distance.

6. The apparatus of claim 1, wherein a surface of the retention portion of the first elongate member is aligned with a surface of a central portion of the first elongate member when the retention portion is in the first configuration, the surface of the retention portion of the first elongate member is out of alignment with the surface of the central portion of the first elongate member when the retention portion is in the second configuration.

7. The apparatus of claim 1, wherein the second elongate member defines a lumen therethrough.

8. The apparatus of claim 1, wherein a proximal end portion of the second elongate member includes an engagement portion configured to be removably coupled to an insertion tool such that movement of the insertion tool along a longitudinal axis of the second elongate member results in movement of the second elongate member along the longitudinal axis.

9. The apparatus of claim 1, wherein the central portion of the second elongate member is configured to move in a proximal direction within the first elongate member when the retention portion of the first elongate member is moved from the first configuration to the second configuration.

10. The apparatus of claim 1, wherein the proximal end portion of the first elongate member includes a shoulder configured to engage a proximal end portion of the second elongate member to limit movement of the central portion of the second elongate member within the first elongate member.

11. The apparatus of claim 1, wherein the second elongate member is substantially coaxial with the first elongate member.

12. An apparatus, comprising:
a first elongate member including a retention portion having a first size when in a first configuration and a second size when in a second configuration, the second size different from the first size;
a second elongate member having at least a portion disposed within the first elongate member, the second elongate member including an actuation portion configured to move the retention portion of the first elongate member from the first configuration to the second configuration when the portion of the second elongate member is moved within the first elongate member; and
a nut threadedly coupled to the first elongate member.

13. The apparatus of claim 12, wherein the retention portion of the first elongate member is configured to deform when moved from the first configuration to the second configuration.

14. The apparatus of claim 12, wherein the retention portion includes a first surface and a second surface, the first surface of the retention portion is substantially normal to a longitudinal axis of the first elongate member when the retention portion is in the second configuration, the first surface of the retention portion is substantially parallel to the second surface of the retention portion when the retention portion is in the second configuration.

15. The apparatus of claim 12, wherein:
the first size is in a radial direction, the first size being substantially equal to an outer diameter of a distal end portion of the first elongate member; and
the second size is in the radial direction, the second size being greater than the outer diameter of the distal end portion of the first elongate member.

16. The apparatus of claim 12, wherein:
the retention portion is at a distal end portion of the first elongate member; and
the nut is threadedly coupled to a proximal end portion of the first elongate member.

17. The apparatus of claim 12, wherein a proximal end portion of the second elongate member includes an engagement portion configured to be removably coupled to an insertion tool such that movement of the insertion tool along a longitudinal axis of the second elongate member results in movement of the second elongate member along the longitudinal axis.

18. The apparatus of claim 12, wherein the portion of the second elongate member is configured to move in a proximal direction within the first elongate member when the retention portion of the first elongate member is moved from the first configuration to the second configuration.

19. A method, comprising:
inserting a bone fixation device into a body, the bone fixation device including an elongate member and a nut threadedly coupled to the elongate member;
disposing at least a portion of the elongate member within a passageway defined a bone structure;
deforming a retention portion of the elongate member such that a surface of the retention portion of the elongate member is in contact with a first external surface of the bone structure; and
moving the nut relative to the elongate member such that a surface of the nut is in contact with a second external surface of the bone structure.

20. The method of claim 19, wherein:
the elongate member is a first elongate member; and
the deforming includes moving at least a portion of a second elongate member proximally within the first elongate member.

21. The method of claim 19, wherein:
the bone structure is a first bone structure, the passageway being defined within the first bone structure and a second bone structure, the first bone structure is a inferior vertebra, the second bone structure is a superior vertebra; and
the disposing includes disposing the portion of the elongate member within the passageway defined by the inferior vertebra and the superior vertebra such that a central portion of the elongate member crosses a facet joint between the inferior vertebra and the superior vertebra.

22. The method of claim 19, wherein:
the bone structure is a first bone structure, the passageway being defined within the first bone structure and a second bone structure, the first bone structure is a inferior vertebra, the second bone structure is a superior vertebra;
the disposing includes disposing the portion of the elongate member within the passageway defined by the inferior vertebra and the superior vertebra such that a central portion of the elongate member crosses a facet joint between the inferior vertebra and the superior vertebra; and
the deforming and the moving are performed such that movement of the inferior vertebra relative to the superior vertebra is limited.

23. The method of claim 19, wherein:
the moving is performed independently from the deforming.

24. The method of claim 19, wherein:
the inserting includes inserting the bone fixation device via a lateral incision;
the bone structure is a first bone structure, the passageway being defined within the first bone structure and a second bone structure, the first bone structure is a inferior vertebra, the second bone structure is a superior vertebra; and
the disposing includes moving the bone fixation device in a lateral-to-medial direction within the pathway such that a central portion of the elongate member crosses a facet joint between the inferior vertebra and the superior vertebra.

25. The method of claim 19, wherein:
the disposing includes disposing the portion of the elongate member within the passageway such that the nut and the retention portion are outside of the passageway.

26. The method of claim 19, wherein the moving includes moving the nut relative to the elongate member in a first direction, the method further comprising:
moving the nut relative to the elongate member in a second direction opposite the first direction.

27. The method of claim 19, wherein the elongate member is a first elongate member, the deforming including moving at least a portion of a second elongate member proximally within the first elongate member, the method further comprising:
removing the second elongate member from the first elongate member after the deforming.

28. The method of claim 19, wherein the elongate member is a first elongate member, the deforming including moving at least a portion of a second elongate member proximally within a lumen defined by the first elongate member, the method further comprising:
removing the second elongate member from the lumen of the first elongate member after the deforming; and
conveying a bone graft material into the lumen of the first elongate member after the removing.
